(19) United States
(12) Patent Application Publication
(10) Pub. No.: US 2025/0108437 A1
(43) Pub. Date: Apr. 3, 2025

(54) SYSTEM FOR BODY FLUID ISOMER ANALYSIS

(71) Applicant: KONINKLIJKE PHILIPS N.V., EINDHOVEN (NL)

(72) Inventors: DIMITRI GEORGE KOSTAKIS, ORLANDO, FL (US); DONALD ALAN FORRER JR., ORLANDO, FL (US); DAVID COCCO, ORLANDO, FL (US)

(21) Appl. No.: 18/834,274

(22) PCT Filed: Jan. 17, 2023
(86) PCT No.: PCT/EP2023/050911

§ 371 Date: Jul. 30, 2024

Related U.S. Application Data
(60) Provisional application No. 63/305,822, filed on Feb. 2, 2022.

(51) Int. Cl. B22F 10/80; B22F 10/28; B33Y 10/00; B33Y 50/00; B33Y 70/00; G16C 60/00; H05K 9/00

(52) U.S. Cl. CPC B22F 10/80; B22F 10/28; G16C 60/00; B33Y 10/00; B33Y 50/00; B33Y 70/00; H05K 9/0075

(57) ABSTRACT

Systems and methods for additively manufacturing magnetic shielding components, such as magnetic shielding components for use in magnetic resonant (MR) environments, are provided. The methods includes selecting a printing material based on one or more magnetic properties and/or one or more manufacturing properties. The method further includes manufacturing a magnetic shielding component from the printing material. The magnetic shielding component is configured to block a magnetic field having a field strength between about 0.7 and 7.0 Tesla. The methods further include creating a unique material for additive manufacturing by evaluating and optimizing a plurality of materials. The optimized material is then used to additively manufacture a magnetic shielding component configured to block magnetic fields generated in an MR environment.

### FIELD OF THE DISCLOSURE

The present disclosure is directed generally to systems and methods for additively manufacturing magnetic shielding components.

### BACKGROUND

Magnetic shielding of electromechanical components is typically executed by bending, machining, or otherwise mechanically forming shielding components from materials with specific magnetic permeability values. Permeability is defined as the relative increase or decrease in a magnetic field within the material in response to an applied magnetic field. Magnetic resonant (MR) environments present unique design needs due to the strong and static magnetic field generated during operation. Electromechanical components operating within the MR environment must be able to function at an acceptable level while this magnetic field is being generated. This level of performance is difficult to achieve unless the electromechanical components are shielded sufficiently. The quality of the shielding depends both on the properties of the material forming the shielding component, as well as the geometry of the shielding component itself.

The most common issues with shielding components arise due to manufacturing defects and limitations on shielding geometries. While simple shapes such as cylinders can be easily manufactured, linking shielding components together without loss of performance is challenging. The majority of production is currently realized through hand-machining in workshops which limits the creation of complicated geometries. In the MR environment, this limitation on geometries can reduce shielding effectiveness and can lead to packaging obstacles for component placement, cable routing, and efficient shield design. Accordingly, there is a need in the art for improved systems and methods for manufacturing magnetic shielding components suitable for use in the MR environment.

### SUMMARY OF THE DISCLOSURE

The present disclosure is directed generally to systems and methods for additively manufacturing magnetic shielding components, such as magnetic shielding components for use in magnetic resonant (MR) environments. The methods include creating a unique material for additive manufacturing by evaluating and optimizing a plurality of materials. The optimized material is then used to additively manufacture a magnetic shielding component.

Generally, in one aspect, a method for additively manufacturing a magnetic shielding component for use in an MR system is provided. The method includes selecting a printing material based on one or more magnetic properties and/or one or more manufacturing properties.

The method includes manufacturing a magnetic shielding component from the printing material. The magnetic shielding component is configured to block a magnetic field having a field strength between about 0.7 and 7.0 Tesla.

The manufacturing may be performed via laser powder bed fusion, direct metal laser sintering, or selective laser melting. The magnetic shielding component may include one or more complex geometries. The one or more complex geometries may include at least one of multi-layered shielding, one piece shielding, concentric cylinders, large contours, and complex contours.

Generally, in another aspect, a method of selecting and optimizing a unique composition is disclosed. The unique composition is for additively manufacturing a magnetic shielding component. The method includes selecting a plurality of bulk-form materials. The plurality of bulk-form materials are selected based on one or more magnetic properties. The one or more material properties may include at least one of permeability and magnetic attraction. The bulk-form materials may be metallic alloys formed as discs or plates.

The method further includes conducting a single track laser scan of each of the plurality of bulk-form materials. The single track laser scan may be performed via a laser powder bed fusion laser.

The method further includes evaluating one or more bulk-form properties of each of the plurality of bulk-form materials formed as a result of the single track laser scan.

The method further includes selecting a subset of the plurality of bulk-form materials. The subset is selected based on the evaluated bulk-form properties. The one or more bulk-form properties may include at least one of laser absorption, laser penetration, laser-materials interaction, rapid solidification, and thermal stress relief.

The method further includes obtaining one or more powders. Each of the one or more powders corresponds to one of the bulk-form materials of the subset.

The method further includes modifying a composition of each of the one or more powders. The powders are modified based on one or more optimized composition parameters. The one or more optimized composition parameters may include at least one of permeability and magnetic attraction.

The method further includes forming samples of each of the one or more powders. The samples may be formed via laser powder bed fusion.

The method further includes determining a sample characterization of each of the samples. The sample characterization is based on one or more sample defects created in the samples during forming. The one or more sample defects may include at least one of keyhole porosity, solidification cracking, balling, and lack-of-fusion flaws

The method further includes selecting, based on the sample characterizations, the composition of one of the one or more samples as the unique composition for additively manufacturing the shielding component.

According to an example, the method may further include manufacturing the magnetic shielding component using the unique composition according to one or more optimized manufacturing parameters. The one or more optimized manufacturing parameters may include at least one of laser power, scan speed, hatch spacing, and powder bed thickness.

According to an example, the method may further include determining, based on the sample characterizations, the one or more optimized manufacturing parameters.

In various implementations, a processor or controller may be associated with one or more storage media (generically referred to herein as “memory,” e.g., volatile and non-volatile computer memory such as RAM, PROM, EPROM, EEPROM, floppy disks, compact disks, optical disks, magnetic tape, SSD, etc.). In some implementations, the storage media may be encoded with one or more programs that, when executed on one or more processors and/or controllers, perform at least some of the functions discussed herein. Various storage media may be fixed within a processor or controller or may be transportable, such that the one or more programs stored thereon can be loaded into a processor or controller so as to implement various aspects as discussed herein. The terms “program” or “computer program” are used herein in a generic sense to refer to any type of computer code (e.g., software or microcode) that can be employed to program one or more processors or controllers.

It should be appreciated that all combinations of the foregoing concepts and additional concepts discussed in greater detail below (provided such concepts are not mutually inconsistent) are contemplated as being part of the inventive subject matter disclosed herein. In particular, all combinations of claimed subject matter appearing at the end of this disclosure are contemplated as being part of the inventive subject matter disclosed herein. It should also be appreciated that terminology explicitly employed herein that also may appear in any disclosure incorporated by reference should be accorded a meaning most consistent with the particular concepts disclosed herein.

These and other aspects of the various embodiments will be apparent from and elucidated with reference to the embodiment(s) described hereinafter.

### BRIEF DESCRIPTION OF THE DRAWINGS

In the drawings, like reference characters generally refer to the same parts throughout the different views. Also, the drawings are not necessarily to scale, emphasis instead generally being placed upon illustrating the principles of the various embodiments.

FIG. 1A is a cross-sectional view of a machined magnetic shielding component, in accordance with an example.

FIG. 1B is a cross-sectional view of an additively manufactured magnetic shielding component, in accordance with an example.

FIG. 2A is an isometric view of a bended-sheet metal magnetic shielding component, in accordance with an example.

FIG. 2B is an isometric view of an additively manufactured magnetic shielding component, in accordance with an example.

FIG. 3 is a flow chart of an additively manufacturing a magnetic shielding component for use in a magnetic resonant (MR) system, in accordance with an example.

FIG. 4 is a flow chart of a method for selecting and optimizing a unique composition for additively manufacturing a magnetic shielding component, in accordance with an example.

### DETAILED DESCRIPTION OF EMBODIMENTS

The present disclosure is directed generally to systems and methods for additively manufacturing magnetic shielding components, such as magnetic shielding components for use in magnetic resonant (MR) environments. The methods include creating a unique material for additive manufacturing by evaluating and optimizing a plurality of materials. The optimized material is then used to additively manufacture a magnetic shielding component configured to block magnetic fields generated in an MR environment.

The creating of the unique material is a multi-step process. First, a number (such as ten) of materials are selected to be analyzed in bulk-form. These chosen materials exhibit high magnetic permeability (such as around 10,000, but preferably greater than 20,000) and low magnetic attraction. The selected materials are typically metallic alloys, and may include one or more of the following: 1018 low-carbon steel, annealed 4140 medium-carbon steel, MuMetal® (for example, 77% nickel, 16% iron, 5% copper, and 2% chromium), high nickel steel, Netic® (pure iron), permalloy (for example, 45% nickel, 55% iron), molybdenum permalloy (for example, 79% nickel, 17% iron, 2% molybdenum), supermalloy (for example, 79% nickel, 16% iron, 2% molybdenum), Hipernik™ (50% nickel, 50% iron). The selected materials may also include bulk metal glasses, such as iron-phosphorous-carbon, iron-silicon-boron-phosphorous, and/or iron-boron-yttrium-based bulk metal glasses.

The selected bulk-form materials are typically acquired in disc-or plate-form. The selected bulk-form materials are then evaluated via single track laser scanning. In single track laser scanning, a laser scans the bulk-form materials at various speeds and power. The laser scan may lead to defects on the bulk-form material, such as keyhole porosity, solidification cracking, balling, and/or lack-of-fusion flaws. Following the scanning, the bulk-form materials are evaluated for various properties, such as laser absorption, laser penetration, laser-material interaction, rapid solidification, and thermal stress relief.

Based on the evaluation of the bulk-form properties, a subset (such as three) of bulk-form materials are selected for further analysis. The selected materials typically exhibit superior performance under single track laser scanning compared to the other bulk-form materials. For example, the selected materials may exhibit superior rapid solidification, resulting in minimal defects on the bulk-form material.

Next, powders are obtained corresponding to the selected bulk-form materials. The powders may be obtained from a third party, or they may be the result of pulverizing the selected bulk-form materials. The composition of the powders are then modified according to optimization parameters such as permeability and magnetic attraction, resulting in one or more unique compositions.

The unique powders are then formed into one or more samples. The samples are formed via additive manufacturing, such as laser powder bed fusion (LPBF) to characterize the buildability and/or printability of the powders. Further, the samples are also formed using various laser powers, scan speeds, hatch speeds, hatch spacing, and powder bed thickness to both evaluate the powders, as well as to optimize the parameters of the additive manufacturing process. Once the samples are formed and characterized, the unique composition of the best performing sample is selected as the unique material to additively manufacture a shielding component.

Once the unique material has been chosen, the magnetic shielding component is created using additive manufacturing. The additive manufacturing process can be LPBF, direct metal laser sintering (DMLS), or selective laser melting (SLM). The parameters of the additive manufacturing process may be configured according to the aforementioned optimized parameters. The formed magnetic shielding component can include one or more complex geometries, such as multi-layered shielding, one piece shielding, concentric cylinders, and/or complex contours. The magnetic shielding component is configured to block a magnetic field having a field strength between 0.7 and 7.0 Tesla in order to protect electro-mechanical components in an MR environment.

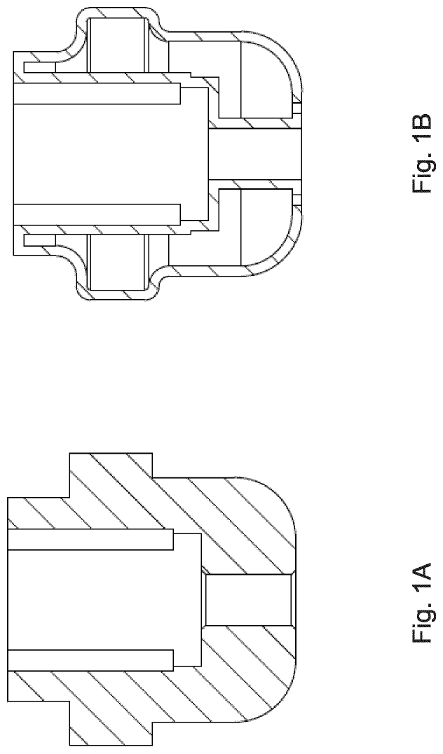

FIG. 1A is a cross-sectional view of a machined magnetic shielding component. This magnetic shielding component is created by cutting a bulk form material into the shape shown in FIG. 1A. The cutting may be performed through a variety of techniques and/or equipment.

FIG. 1B is a cross-sectional view of an additively manufactured magnetic shielding component. As seen in FIG. 1B, the additively manufacturing magnetic shielding component includes a number of more complicated geometric features than the component of FIG. 1A. These complicated geometric features allow for the manufacturing of a lighter weight component, as well as the ability to customize the wall thickness for the particular MR application. For example, the wall thickness may be customized based on the strength of the magnetic field generated by an MR device. In one example, this magnetic field can have a strength of 0.7 to 7.0 Tesla.

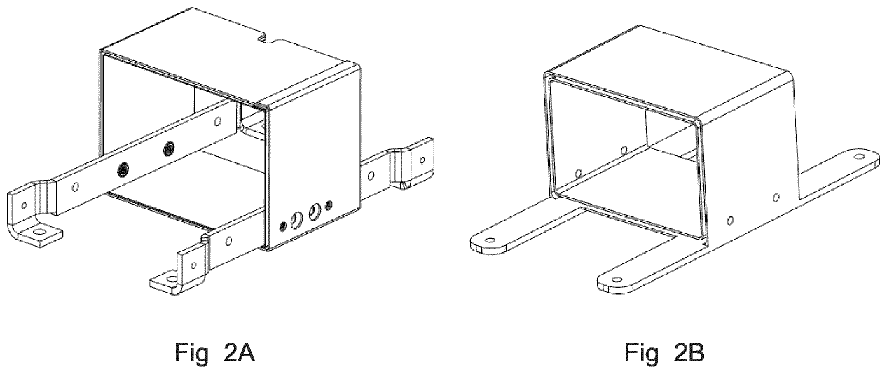

FIG. 2A is an isometric view of a bended-sheet metal magnetic shielding component, while FIG. 2B is an isometric view of an additively manufactured magnetic shielding component. As demonstrated in FIGS. 2A and 2B, the bended-sheet metal magnetic shielding component requires the assembly of several sub-components. This assembly may require the machining of several holes and attaching the sub-components with fasteners, as well as potentially welding the sub-components together. This machining, fastening, and welding can result in lengthening the time of the manufacturing process, as well as introducing the possibility of pitting or other defects. On the contrary, additively manufacturing magnetic shielding components does not require these additional steps, as the entire component is a single piece formed by additive manufacturing. In the cases of FIGS. 1B and 2B, the component can be manufactured from a metallic alloy using LPBF, DMLS, or SLM.

A series of evaluations are performed to determine the metallic alloy to use as the additive manufacturing material. First, a number of metallic alloys are selected to be tested in their bulk-form. These metallic alloys are selected due to their high magnetic shielding capabilities quantified by magnetic permeability in the order of 10,000, preferably greater than 20,000. These metallic alloys may include 1018 low-carbon steel, annealed 4140 medium-carbon steel, MuMetal® (for example, 77% nickel, 16% iron, 5% copper, and 2% chromium), high nickel steel, Netic® (pure iron), permalloy (for example, 45% nickel, 55% iron), molybdenum permalloy (for example, 79% nickel, 17% iron, 2% molybdenum), supermalloy (for example, 79% nickel, 16% iron, 5% molybdenum), Hipernik™ (50% nickel, 50% iron). The selected materials may also include bulk metal glasses, such as iron-phosphorous-carbon, iron-silicon-boron-phosphorous, and/or iron-boron-yttrium-based bulk metal glasses. The selected bulk-form materials are typically selected in their disc-or plate-form.

The selected bulk-form materials are analyzed for buildability and printability using the additive manufacturing methods listed above, such as LBPF. The starting composition, phase constituents, and microstructure of the selected bulk-form materials are characterized by scanning electron microscopy (SEM) equipped with energy dispersive spectroscopy (XEDS) and x-ray diffraction. The bulk-form materials are then subjected to a single track laser scan study. In single track laser scan, the laser of the additive manufacturing method (such as LBPF) is scanned on the surfaces of the bulk-form materials at various powers and scan speeds. At a constant power, varying the scan speed results in variations of melt pool development characteristics.

Scanning the bulk-form materials may result in the formation of a number of defects. These defects can include cracking, keyholing, balling, and lack-of-fusion flaws. Both cracking and keyholing may be the result of excessive laser power incident on the bulk-form materials. This excessive power may be due to slow scan speeds. Balling and lack-of-fusion flaws may be the result of insufficient laser power incident on the bulk-form materials, potentially due to high scan speeds.

Based on the scanning results, several bulk-form properties can be evaluated, such as laser absorption or penetration (such as keyholing), laser-materials interaction (such as melting and potential differential evaporation), rapid solidification (such as compositional portioning and microstructural development including keyhole porosity, solidification cracking, and lack-of-fusion flaws), and thermal stress relief. Based on these properties, a subset of the strongest performing materials are chosen for further analysis. In one example, the subset includes three materials.

Powder forms of the selected materials are then obtained. The chosen additive manufacturing method (LBPF, DMLS, SLM) is then used to create a plurality of samples from each powder using a variety of manufacturing parameter settings, such as scan speed, laser power, hatch spacing, and powder bed thickness. Analyzing the additively manufactured samples serves two purposes. First, the samples are analyzed to determine the buildability and/or printability of the powders themselves, as additive manufacturing using the powders may result in additional characteristics or defects not found in the initial bulk-form analysis. Second, the samples are analyzed to determine the optimum manufacturing parameters settings of the additive manufacturing method.

In one example, additively manufactured samples were manufactured using LBPF from a metallic alloy. In this example, the samples were manufactured at a constant power over varying scan speeds (300 to 1500 millimeters per second). However, it was found that laser scan speeds of 300 and 500 millimeters per second results in the formation of keyhole pores. This formation of keyhole pores may be due to excessive laser power input due to the slower scan speed. Further, laser scan speed of 1100, 1300, and 1500 millimeters per second result in the formation of lack-of-fusion flaws. The lack-of-fusion flaws may be the result of insufficient laser power input due to the faster scan speeds. Accordingly, optimum scan speeds for additive manufacturing using this powder are 700 and 900 millimeters per second. It is further noteworthy that none of the various scan speeds result in the solidification cracking. Accordingly, by analyzing several different samples using a variety of manufacturing parameter settings, both optimum materials and manufacturing parameters may be identified.

Following the sample analysis, three-dimensional mechanical test components can be produced using the one or more of the powders. Mechanical test samples may undergo a series of mechanical stress tests to evaluate the buildability and/or printability of the corresponding powder, as well as the manufacturing parameter settings. Further, magnetic permeability testing may be conducted to evaluate the shielding properties of the mechanical test components. For example, the mechanical test components may be evaluated to determine if they are capable of blocking magnetic fields in MR environments, such as magnetic fields with strengths between 0.7 and 7.0 Tesla.

Further, based on either the sample evaluation or the mechanical test sample evaluation, the composition of the powders themselves may be modified to create a unique composition. Modifying the composition of the powders may be done to improve printability, reduce defects, improve magnetic shielding properties, reduce manufacturing and/or material cost, or for wide variety of additional purposes. Modifying the composition can include a number of processes, such as adjusting the ratios of the elements currently comprising the composition. For example, modifying the composition can include reducing the amount of nickel in permalloy. Additionally, modifying the composition can include adding a new element to the existing composition. The new element may be metal, metalloid, or nonmetal. Following the modification of the composition, new samples may be manufactured to analyze the characteristics of the new, unique composition. This may be an iterative process resulting in several incremental modifications of the composition until a satisfactory composition (based on sample and mechanical test component evaluation) is produced.

A wide array of components are capable of being manufactured using additive manufacturing. Many of these components include complex geometries. These complex geometries may be designed using three-dimensional modelling software. Modelling software may be also capable of performing simulations and/or analyses of the shielding properties of the complex geometries.

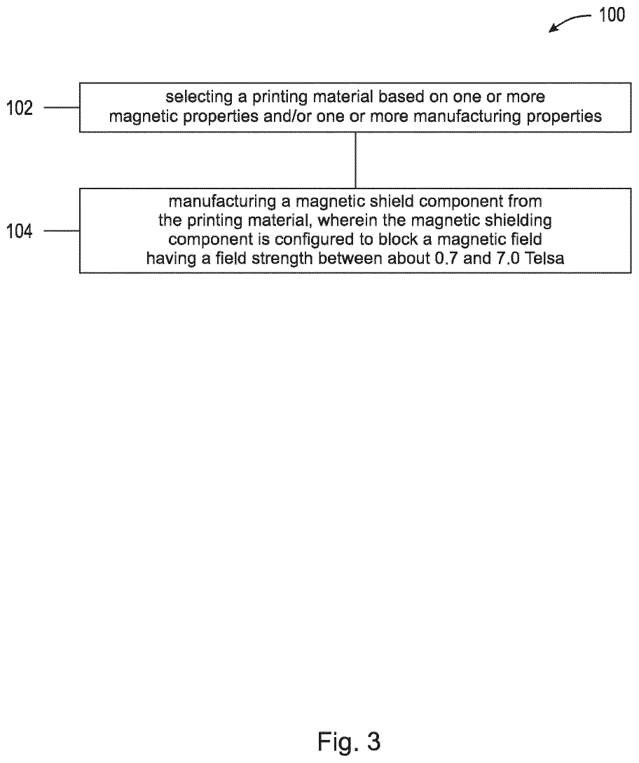

FIG. 3 is a flowchart of a method 100 for additively manufacturing a magnetic shielding component for use in an MR system. The method 100 includes selecting 102 a printing material based on one or more magnetic properties and/or one or more manufacturing properties.

The method 100 further includes manufacturing 104 a magnetic shielding component from the printing material. The magnetic shielding component is configured to block a magnetic field having a field strength between about 0.7 Tesla and about 7.0 Tesla.

The manufacturing may be performed via LBPF, DMLS, or SLM. The magnetic shielding component may include one or more complex geometries. The one or more complex geometries may include at least one of multi-layered shielding, one piece shielding, concentric cylinders, large contours, and complex contours.

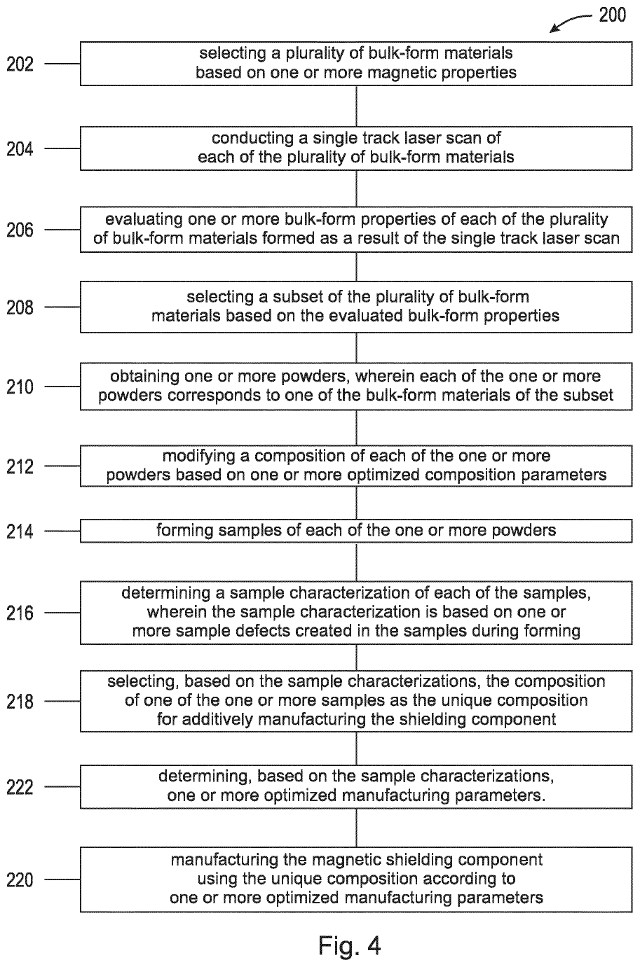

FIG. 4 is a flowchart of a method 200 of selecting and optimizing a unique composition. The unique composition is for additively manufacturing a magnetic shielding component. The method 200 includes selecting 202 a plurality of bulk-form materials. The plurality of bulk-form materials are selected based on one or more magnetic properties. The one or more material properties may include at least one of permeability and magnetic attraction. The bulk-form materials may be metallic alloys formed as discs or plates.

The method 200 further includes conducting 204 a single track laser scan of each of the plurality of bulk-form materials. The single track laser scan may be performed via a laser powder bed fusion laser.

The method 200 further includes evaluating 206 one or more bulk-form properties of each of the plurality of bulk-form materials formed as a result of the single track laser scan.

The method 200 further includes selecting 208 a subset of the plurality of bulk-form materials. The subset is selected based on the evaluated bulk-form properties. The one or more bulk-form properties may include at least one of laser absorption, laser penetration, laser-materials interaction, rapid solidification, and thermal stress relief.

The method 200 further includes obtaining 210 one or more powders. Each of the one or more powders corresponds to one of the bulk-form materials of the subset.

The method 200 further includes modifying 212 a composition of each of the one or more powders. The powders are modified based on one or more optimized composition parameters. The one or more optimized composition parameters may include at least one of permeability and magnetic attraction.

The method 200 further includes forming 214 samples of each of the one or more powders. The samples may be formed via laser powder bed fusion.

The method 200 further includes determining 216 a sample characterization of each of the samples. The sample characterization is based on one or more sample defects created in the samples during forming. The one or more sample defects may include at least one of keyhole porosity, solidification cracking, balling, and lack-of-fusion flaws

The method 200 further includes selecting 218, based on the sample characterizations, the composition of one of the one or more samples as the unique composition for additively manufacturing the shielding component. According to an example, the method 200 may further include determining 222, based on the sample characterizations, the one or more optimized manufacturing parameters. According to a further example, the method 200 may further include manufacturing 220 the magnetic shielding component using the unique composition according to one or more optimized manufacturing parameters. The one or more optimized manufacturing parameters may include at least one of laser power, scan speed, hatch spacing, and powder bed thickness.

All definitions, as defined and used herein, should be understood to control over dictionary definitions, definitions in documents incorporated by reference, and/or ordinary meanings of the defined terms.

The indefinite articles “a” and “an,” as used herein in the specification and in the claims, unless clearly indicated to the contrary, should be understood to mean “at least one.”

The phrase “and/or,” as used herein in the specification and in the claims, should be understood to mean “either or both” of the elements so conjoined, i.e., elements that are conjunctively present in some cases and disjunctively present in other cases. Multiple elements listed with “and/or” should be construed in the same fashion, i.e., “one or more” of the elements so conjoined. Other elements may optionally be present other than the elements specifically identified by the “and/or” clause, whether related or unrelated to those elements specifically identified.

As used herein in the specification and in the claims, “or” should be understood to have the same meaning as “and/or” as defined above. For example, when separating items in a list, “or” or “and/or” shall be interpreted as being inclusive, i.e., the inclusion of at least one, but also including more than one, of a number or list of elements, and, optionally, additional unlisted items. Only terms clearly indicated to the contrary, such as “only one of” or “exactly one of,” or, when used in the claims, “consisting of,” will refer to the inclusion of exactly one element of a number or list of elements. In general, the term “or” as used herein shall only be interpreted as indicating exclusive alternatives (i.e. “one or the other but not both”) when preceded by terms of exclusivity, such as “either,” “one of,” “only one of,” or “exactly one of.”

As used herein in the specification and in the claims, the phrase “at least one,” in reference to a list of one or more elements, should be understood to mean at least one element selected from any one or more of the elements in the list of elements, but not necessarily including at least one of each and every element specifically listed within the list of elements and not excluding any combinations of elements in the list of elements. This definition also allows that elements may optionally be present other than the elements specifically identified within the list of elements to which the phrase “at least one” refers, whether related or unrelated to those elements specifically identified.

It should also be understood that, unless clearly indicated to the contrary, in any methods claimed herein that include more than one step or act, the order of the steps or acts of the method is not necessarily limited to the order in which the steps or acts of the method are recited.

In the claims, as well as in the specification above, all transitional phrases such as “comprising,” “including,” “carrying,” “having,” “containing,” “involving,” “holding,” “composed of,” and the like are to be understood to be open-ended, i.e., to mean including but not limited to. Only the transitional phrases “consisting of” and “consisting essentially of” shall be closed or semi-closed transitional phrases, respectively.

The above-described examples of the described subject matter can be implemented in any of numerous ways. For example, some aspects may be implemented using hardware, software or a combination thereof. When any aspect is implemented at least in part in software, the software code can be executed on any suitable processor or collection of processors, whether provided in a single device or computer or distributed among multiple devices/computers.

The present disclosure may be implemented as a system, a method, and/or a computer program product at any possible technical detail level of integration. The computer program product may include a computer readable storage medium (or media) having computer readable program instructions thereon for causing a processor to carry out aspects of the present disclosure.

The computer readable storage medium can be a tangible device that can retain and store instructions for use by an instruction execution device. The computer readable storage medium may be, for example, but is not limited to, an electronic storage device, a magnetic storage device, an optical storage device, an electromagnetic storage device, a semiconductor storage device, or any suitable combination of the foregoing. A non-exhaustive list of more specific examples of the computer readable storage medium includes the following: a portable computer diskette, a hard disk, a random access memory (RAM), a read-only memory (ROM), an erasable programmable read-only memory (EPROM or Flash memory), a static random access memory (SRAM), a portable compact disc read-only memory (CD-ROM), a digital versatile disk (DVD), a memory stick, a floppy disk, a mechanically encoded device such as punch-cards or raised structures in a groove having instructions recorded thereon, and any suitable combination of the foregoing. A computer readable storage medium, as used herein, is not to be construed as being transitory signals per se, such as radio waves or other freely propagating electromagnetic waves, electromagnetic waves propagating through a waveguide or other transmission media (e.g., light pulses passing through a fiber-optic cable), or electrical signals transmitted through a wire.

Computer readable program instructions described herein can be downloaded to respective computing/processing devices from a computer readable storage medium or to an external computer or external storage device via a network, for example, the Internet, a local area network, a wide area network and/or a wireless network. The network may comprise copper transmission cables, optical transmission fibers, wireless transmission, routers, firewalls, switches, gateway computers and/or edge servers. A network adapter card or network interface in each computing/processing device receives computer readable program instructions from the network and forwards the computer readable program instructions for storage in a computer readable storage medium within the respective computing/processing device.

Computer readable program instructions for carrying out operations of the present disclosure may be assembler instructions, instruction-set-architecture (ISA) instructions, machine instructions, machine dependent instructions, microcode, firmware instructions, state-setting data, configuration data for integrated circuitry, or either source code or object code written in any combination of one or more programming languages, including an object oriented programming language such as Smalltalk, C++, or the like, and procedural programming languages, such as the “C” programming language or similar programming languages. The computer readable program instructions may execute entirely on the user's computer, partly on the user's computer, as a stand-alone software package, partly on the user's computer and partly on a remote computer or entirely on the remote computer or server. In the latter scenario, the remote computer may be connected to the user's computer through any type of network, including a local area network (LAN) or a wide area network (WAN), or the connection may be made to an external computer (for example, through the Internet using an Internet Service Provider). In some examples, electronic circuitry including, for example, programmable logic circuitry, field-programmable gate arrays (FPGA), or programmable logic arrays (PLA) may execute the computer readable program instructions by utilizing state information of the computer readable program instructions to personalize the electronic circuitry, in order to perform aspects of the present disclosure.

Aspects of the present disclosure are described herein with reference to flowchart illustrations and/or block diagrams of methods, apparatus (systems), and computer program products according to examples of the disclosure. It will be understood that each block of the flowchart illustrations and/or block diagrams, and combinations of blocks in the flowchart illustrations and/or block diagrams, can be implemented by computer readable program instructions.

The computer readable program instructions may be provided to a processor of a, special purpose computer, or other programmable data processing apparatus to produce a machine, such that the instructions, which execute via the processor of the computer or other programmable data processing apparatus, create means for implementing the functions/acts specified in the flowchart and/or block diagram block or blocks. These computer readable program instructions may also be stored in a computer readable storage medium that can direct a computer, a programmable data processing apparatus, and/or other devices to function in a particular manner, such that the computer readable storage medium having instructions stored therein comprises an article of manufacture including instructions which implement aspects of the function/act specified in the flowchart and/or block diagram or blocks.

The computer readable program instructions may also be loaded onto a computer, other programmable data processing apparatus, or other device to cause a series of operational steps to be performed on the computer, other programmable apparatus or other device to produce a computer implemented process, such that the instructions which execute on the computer, other programmable apparatus, or other device implement the functions/acts specified in the flowchart and/or block diagram block or blocks.

The flowchart and block diagrams in the Figures illustrate the architecture, functionality, and operation of possible implementations of systems, methods, and computer program products according to various examples of the present disclosure. In this regard, each block in the flowchart or block diagrams may represent a module, segment, or portion of instructions, which comprises one or more executable instructions for implementing the specified logical function(s). In some alternative implementations, the functions noted in the blocks may occur out of the order noted in the Figures. For example, two blocks shown in succession may, in fact, be executed substantially concurrently, or the blocks may sometimes be executed in the reverse order, depending upon the functionality involved. It will also be noted that each block of the block diagrams and/or flowchart illustration, and combinations of blocks in the block diagrams and/or flowchart illustration, can be implemented by special purpose hardware-based systems that perform the specified functions or acts or carry out combinations of special purpose hardware and computer instructions.

Other implementations are within the scope of the following claims and other claims to which the applicant may be entitled.

While various examples have been described and illustrated herein, those of ordinary skill in the art will readily envision a variety of other means and/or structures for performing the function and/or obtaining the results and/or one or more of the advantages described herein, and each of such variations and/or modifications is deemed to be within the scope of the examples described herein. More generally, those skilled in the art will readily appreciate that all parameters, dimensions, materials, and configurations described herein are meant to be exemplary and that the actual parameters, dimensions, materials, and/or configurations will depend upon the specific application or applications for which the teachings is/are used. Those skilled in the art will recognize, or be able to ascertain using no more than routine experimentation, many equivalents to the specific examples described herein. It is, therefore, to be understood that the foregoing examples are presented by way of example only and that, within the scope of the appended claims and equivalents thereto, examples may be practiced otherwise than as specifically described and claimed. Examples of the present disclosure are directed to each individual feature, system, article, material, kit, and/or method described herein. In addition, any combination of two or more such features, systems, articles, materials, kits, and/or methods, if such features, systems, articles, materials, kits, and/or methods are not mutually inconsistent, is included within the scope of the present disclosure.

1. A method for additively manufacturing a magnetic shielding component for use in a magnetic resonant (MR) system, comprising:
selecting a printing material based on one or more magnetic properties and/or one or more manufacturing properties; and
manufacturing a magnetic shielding component from the printing material, wherein the magnetic shielding component is configured to block a magnetic field having a field strength between about 0.7 Tesla and about 7.0 Tesla.

2. The method of claim 1, wherein the manufacturing is performed via laser powder bed fusion, direct metal laser sintering, or selective laser melting.

3. The method of any claim 1, wherein the magnetic shielding component comprises one or more complex geometries.

4. The method of claim 3, wherein the one or more complex geometries include at least one of multi-layered shielding, one piece shielding, concentric cylinders, large contours, and complex contours.

5. A method of selecting and optimizing a unique composition for additively manufacturing a magnetic shielding component, comprising:
selecting a plurality of bulk-form materials based on one or more magnetic properties;
conducting a single track laser scan of each of the plurality of bulk-form materials;
evaluating one or more bulk-form properties of each of the plurality of bulk-form materials formed as a result of the single track laser scan;
selecting a subset of the plurality of bulk-form materials based on the evaluated bulk-form properties;
obtaining one or more powders, wherein each of the one or more powders corresponds to one of the bulk-form materials of the subset;
modifying a composition of each of the one or more powders based on one or more optimized composition parameters;
forming samples of each of the one or more powders;
determining a sample characterization of each of the samples, wherein the sample characterization is based on one or more sample defects created in the samples during forming; and
selecting, based on the sample characterizations, the composition of one of the one or more samples as the unique composition for additively manufacturing the shielding component.

6. The method of claim 5, wherein the one or more material properties comprise at least one of permeability or magnetic attraction.

7. The method of claim 5, wherein the bulk-form materials metallic alloys formed as discs or plates.

8. The method of claim 5, wherein the single track laser scan is performed via a laser powder bed fusion laser.

9. The method of claim 5, wherein the one or more bulk-form properties comprise at least one of laser absorption, laser penetration, laser-materials interaction, rapid solidification, or thermal stress relief.

10. The method of claim 5, wherein the samples are formed via laser powder bed fusion.

11. The method of claim 5, wherein the one or more optimized composition parameters comprise at least one of permeability or magnetic attraction.

12. The method of claim 5, wherein the one or more sample defects comprise at least one of keyhole porosity, solidification cracking, balling, or lack-of-fusion flaws.

13. The method of claim 5, further comprising manufacturing the magnetic shielding component using the unique composition according to one or more optimized manufacturing parameters.

14. The method of claim 13, further comprising determining, based on the sample characterizations, the one or more optimized manufacturing parameters.

15. The method of claim 14, wherein the one or more optimized manufacturing parameters comprise at least one of laser power, scan speed, hatch spacing, or powder bed thickness.
